(19) United States
(12) Patent Application Publication
(10) Pub. No.: US 2022/0409532 A1
(43) Pub. Date: Dec. 29, 2022

(54) COMPOSITION AND METHOD FOR A TOPICAL TREATMENT FOR VENOM NEUTRALIZATION

(71) Applicant: Brian J. Huber, Rockwall, TX (US)

(72) Inventor: Brian J. Huber, Rockwall, TX (US)

(21) Appl. No.: 17/838,523

(22) Filed: Jun. 13, 2022

Related U.S. Application Data
(60) Provisional application No. 63/214,516, filed on Jun. 24, 2021.

(51) Int. Cl. A61K 9/06; A61K 9/00; A61K 9/107

(52) U.S. Cl. CPC A61K 9/06; A61K 9/0014; A61K 9/107

(57) ABSTRACT

A composition and method which comprises a localized anesthetic into and directly on bite wounds and periodic application of a topical non-toxic formulation that may include a surfactant.

### CROSS-REFERENCE TO RELATED APPLICATIONS

Priority is claimed from U.S. Provisional Patent Application Ser. No. 63/214,516 filed on Jun. 24, 2021, currently pending and which is incorporated by referenced herein.

### BACKGROUND OF THE INVENTION

### 1. Field of the Invention

In general, the present invention is a composition and method for the topical treatment of venom in general. More in particular, the present invention provides a newly advanced topical treatment that is highly effective for venom neutralization utilizing surfactant technology for first aid treatment.

### 2. Description of the Prior Art

It is known that envenomation, from stings and bites, deposits a liquid venom which causes inflammation in human tissue. The inflammation effects range from mild to life-threatening, depending on the amount of the liquid venom resulting from the bite or sting and depending on the human's level of sensitivity to the liquid venom deposited during the bite or sting. It is also known that the time from envenomation to treatment is the key focus to minimize tissue trauma and life-threatening systemic effects of these toxins.

In the prior art, a different mechanism of action compared to a commercial antivenin product such as Inoserp®, Crofab®, and so forth, which are made from immunoglobulin antibodies and are used to treat certain snake venomous bites. Antivenins are recommended only if there are significant clinical symptoms of toxicity or a high risk of toxicity. It is also known that specific antivenins are needed depending on the species of snake, insect, and so forth involved. Antivenin is administered by intravenous injection. Antivenin side effects, such as but not limited to anaphylaxis, may be severe.

Therefore, it is desirable to provide a new approach to mitigating venom toxicities. Topical administration along with a new and improved penetration formulation and method that may provide superior penetration and therefore, provide a more effective delivery of treatment of topical applications for venom neutralization. The above discussed limitations in the prior art are not exhaustive. The current invention provides an inexpensive and effective composition and method use with topical treatments not currently found in the known art.

### SUMMARY OF THE INVENTION

In view of the foregoing disadvantages inherent in the known types of compositions and methods for topical applications in general now present in the prior art, the present invention provides a new and improved effective composition and method of using the same where the prior art fails. As such, the general purpose of the present invention, which will be described subsequently in greater detail, is to provide a new and effective composition and method for topical venom neutralization treatments, which has all the advantages of the prior art and none of the disadvantages.

To attain this, the present invention essentially comprises a localized anesthetic into and directly on bite wounds and periodic application of a topical non-toxic formulation that may include a surfactant such as but not limited to multiple types of surfactants.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in this application to the details of construction, arrangement of the components, and amounts thereof set forth in the following description. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception upon which this disclosure is based may readily be utilized as a basis for the designing of other compositions, methods, and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Therefore, it is an object of the present invention to provide a new and improved composition and method for treating envenomation wherein it is contemplated that the invention may be a broad-spectrum universal venom neutralization formulation that may be applied topically.

An even further object of the present invention is to provide a new and improved composition and method for topical treatments for envenomation susceptible to a low cost of manufacture with regard to ingredients and associated labor of producing same, and, thus accordingly, is then susceptible to low prices of sale to the consuming public thereby making such economically available.

Still another object of the present invention is to provide a new and improved composition and method for topical treatments, which provides all of the advantages of the prior art, while simultaneously overcoming some of the disadvantages normally associated therewith.

Another object of the present invention is to provide a new and improved composition and method for topical treatments that may be used over large areas of skin for those afflicted without or with reduced detrimental side effects.

Yet another object of the present invention is to provide a new and improved composition and method for topical treatments that is commercially available such that public awareness is garnered and those afflicted will have a viable and readily available treatment.

An even further object of the present invention is to provide a new and improved composition and method for topical treatments that combines proven ingredients that have already passed F.D.A. approval and may be sold over the counter.

Still another object of the present invention is to provide a new and improved composition and method for topical treatment conditions in general that may be utilized in a cream, lotion, liquid, gel, and combinations thereof that is non-irritating, fragrance free, and contains no known sensitizing ingredients.

Another object of the current invention is to provide a new and improved composition and method for topical and intralesional treatments that is a venom neutralization formulation for the treatment of bites and stings from but not limited to reptile, jelly fish, scorpion, insect stings and so forth.

These, together with other objects of the invention, along with the various features of novelty, which characterize the invention, are pointed out with particularity in the claims annexed to and forming a part of this disclosure. For a better understanding of the invention, its operating advantages, and the specific objects attained by its uses, reference should be had to the accompanying descriptive matter in which there are illustrated preferred embodiments of the invention.

### DETAILED DESCRIPTION

The present invention may be a formulation utilized as a cream, lotion, liquid, gel, and combinations thereof and may be utilized as topical treatment for venom neutralization. It is also understood that the following description of said components is not limited to exact percentages, quantities, or ingredients and that it is understood equivalent ingredients known in the art may be substituted or added. The invention should be applied only once or twice per day depending on the severity of symptoms. The invention should be applied immediately after the bite or sting and repeated every 5-10 minutes during first few hours post bite. This individual should be seeking proper medical care where available. It is understood that numerous daily applications may be utilized.

In a preferred embodiment, invention may utilize a surfactant. Surfactants are compounds that lower the surface tension between two liquids, between a gas and a liquid, or between a liquid and a solid. Surfactants may act as detergents, wetting agents, emulsifiers, foaming agents, or dispersants.

Surfactants are usually organic compounds that are amphiphilic, meaning they contain both hydrophobic groups and hydrophilic groups. Therefore, a surfactant contains both a water-insoluble (or oil-soluble) component and a water-soluble component. Surfactants will diffuse in water and adsorb at interfaces between air and water or at the interface between oil and water, in the case where water is mixed with oil. The water-insoluble hydrophobic group may extend out of the bulk water phase, into the air or into the oil phase, while the water-soluble head group remains in the water phase.

The invention may be applied topically to the bite wound sites and gently massaged and or rubbed into the skin in the same way a soap or detergent might be used to wash away allergens of any kind. Most liquid venom left behind after a bite or sting is partially oil based. This results in the dispersal of the allergens and or toxins, so as to minimize the concentration of tissue damage resulting from the bite or sting. For best results, the invention should be applied as soon as possible, rubbing into the skin at the location of the bite or sting.

In the case of snake envenomation, the invention should additionally, be applied above and below the sites to cover the areas of “potential” subcutaneous dispersion of the venom. The opportunity and goal are to stop or neutralize the venom at the local site of the bite wound before the systemic circulation of the venom toxins and life-threatening effects occur.

Therefore, the invention contemplates a method for treating a venomous bite wound comprising the steps: providing a pharmaceutically effective amount of surfactant in a composition; and applying said composition to said venomous bite wound.

A number of implementations have been described herein. Nevertheless, it will be understood that various modifications may be made. Accordingly, other implementations are within the scope of the following claims. Changes may be made in the combinations, operations, and arrangements of the various parts, elements, and amounts described herein without departing from the spirit and scope of the invention.

What is claimed:

1. A method for treating a venomous bite wound comprising the steps:
providing a pharmaceutically effective amount of surfactant in a composition; and
applying said composition to said venomous bite wound.
